(19) United States
(12) Patent Application Publication
(10) Pub. No.: US 2025/0072727 A1
(43) Pub. Date: Mar. 6, 2025

(54) ENDOSCOPIC TREATMENT INSTRUMENT

(71) Applicant: OLYMPUS MEDICAL SYSTEMS CORP., Tokyo (JP)

(72) Inventors: Tatsuya SUZUKI, Hachioji-shi (JP); Tomotaka Hayakawa, Kawaguchi-shi (JP); Tomoki Hatsuda, Hino-shi (JP)

(21) Appl. No.: 18/816,639

(22) Filed: Aug. 27, 2024

Related U.S. Application Data
(60) Provisional application No. 63/579,670, filed on Aug. 30, 2023.

(51) Int. Cl. A61B 1/00

(52) U.S. Cl. CPC A61B 1/00142; A61B 1/00137

(57) ABSTRACT

An endoscopic treatment instrument can include a coil sheath, a wire, a wire connection portion, and an end effector. The wire is provided in the coil sheath and configured to move in a longitudinal direction of the coil sheath relative to the coil sheath. The wire connection portion is connected to a distal end side of the wire. The end effector is configured to switch between an open state and closed state in response to movement of the wire, when the end effector is in the closed state, a proximal end of the wire connection portion is located proximally relative to a distal end of the coil sheath.

### CROSS-REFERENCE TO RELATED APPLICATIONS

The present application claims priority based on U.S. Patent Provisional Application No. 63/579,670 filed in the United States on Aug. 30, 2023, the contents of which are incorporated herein by reference.

### TECHNICAL FIELD

The present disclosure relates to an endoscopic treatment instrument.

### BACKGROUND

An endoscopic treatment instrument such as biopsy forceps can be used in endoscopic treatment. Since the endoscopic treatment instrument such as biopsy forceps is a disposable product, it is required to have high performance and low cost.

An example of an endoscopic treatment instrument is described in Japanese Unexamined Patent Application, First Publication No. 2023-064073 (Patent Document 1). The treatment instrument described in Patent Document 1 is an endoscopic treatment instrument that can be into an insertion section of an endoscope. It includes a coil sheath which is formed by winding a metallic wire in a spiral shape, an operation wire which is inserted through an inner space of the coil sheath, forceps which are provided at a distal end portion of the endoscopic treatment instrument, and a support member. The forceps include a first forceps piece and a second forceps piece and are supported by the support member so that the first forceps piece and the second forceps piece are openable and closable. Further, the support member includes a support main body which is fixed to a distal end of the coil sheath and a cylindrical body which is connected to the operation wire and is inserted into an inner space of the support main body.

### SUMMARY

In the endoscopic treatment instrument described in Patent Document 1 and similar treatment tools, the slidability between the endoscopic treatment instrument and a forceps channel may be reduced due to the contact between a rigid member such as the support main body and the forceps channel or the contact between the wire forming the coil sheath and the forceps channel inside (in the forceps channel of) the curved endoscope and hence the operability may be reduced.

In view of the above-described circumstances, the present disclosure provides an endoscopic treatment instrument having improved operability.

An endoscopic treatment instrument can include a coil sheath, a wire, a wire connection portion, and an end effector. The wire is provided in the coil sheath and configured to move in a longitudinal direction of the coil sheath relative to the coil sheath. The wire connection portion is connected to a distal end side of the wire. The end effector is configured to switch between an open state and closed state in response to movement of the wire, when the end effector is in the closed state, a proximal end of the wire connection portion is located proximally relative to a distal end of the coil sheath.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an example of an overall view illustrating an endoscopic treatment system according to an embodiment.

FIG. 2 is an example of an overall view illustrating an endoscopic treatment instrument of the endoscopic treatment system according to an embodiment.

FIG. 3 is an example of a perspective view illustrating a distal end portion of the endoscopic treatment instrument in a closed state according to an embodiment.

FIG. 4 is an example of a perspective view illustrating the distal end portion in an open state according to an embodiment.

FIG. 5 is an example of an exploded perspective view illustrating a treatment section of the endoscopic treatment instrument according to an embodiment.

FIG. 6 is an example of a perspective view illustrating a frame of the treatment section according to an embodiment.

FIG. 7 is an example of a cross-sectional view illustrating the distal end portion in a closed state according to an embodiment.

FIG. 8 is an example of a cross-sectional view illustrating the distal end portion in an open state according to an embodiment.

FIG. 9 is an example of a cross-sectional view illustrating a connection portion between an operation wire and a cylindrical body of the endoscopic treatment instrument according to an embodiment.

FIG. 10 is an example of a cross-sectional view illustrating a wire forming a coil sheath of the endoscopic treatment instrument according to an embodiment.

FIG. 11 is an example of a perspective view illustrating a wire operating section according to a first modified example embodiment.

FIG. 12 is an example of a cross-sectional view illustrating an example of the wire operating section according to an embodiment.

FIG. 13 is an example of a perspective view illustrating an example of an operating section main body according to a second modified example embodiment.

FIG. 14 is an example of a perspective view illustrating an example of the operating section main body according to an embodiment.

FIG. 15 is an example of a perspective view illustrating an example of the operating section main body according to an embodiment.

FIG. 16 is an example of a cross-sectional view illustrating an example of the operating section main body according to an embodiment.

FIG. 17 is an example of a cross-sectional view illustrating the distal end portion in an open state according to a modified example embodiment.

### DETAILED DESCRIPTION

An endoscopic treatment system according to an embodiment of the present disclosure will be described with reference to the drawings. In addition, in each of the following drawings, in order to make each component easier to see, the scale of dimensions may differ depending on the component.

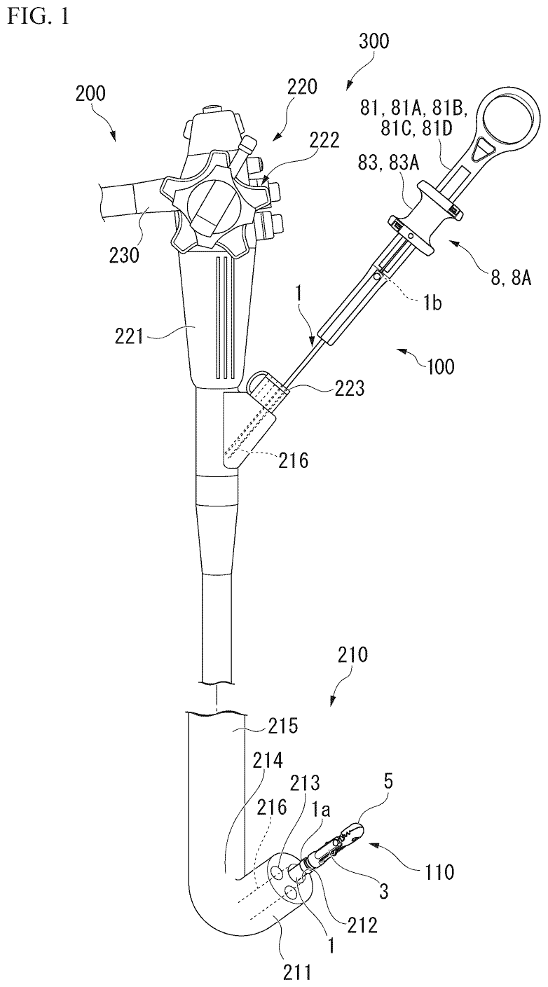

FIG. 1 is an example of an overall view illustrating the endoscopic treatment system 300 according to an embodiment of the present disclosure.

### Endoscopic Treatment System 300

The endoscopic treatment system 300 can include, as illustrated in FIG. 1, an endoscopic treatment instrument 100 and an endoscope 200. The endoscopic treatment instrument 100 can be used by being inserted into the endoscope 200. The endoscopic treatment instrument 100 can comprises a coil sheath 1, a wire 2, a wire connection portion 38, and an end effector 5.

### Endoscope 200

The endoscope 200 can be flexible endoscope and can include an insertion section 210 configured to be inserted into a body from a distal end. An operation section 220 can be attached to a proximal end of the insertion section 210, and a universal cord 230 can be attached to the operation section 220.

The insertion section 210 can be an elongate member that can be inserted into a lumen. The insertion section 210 can include a distal end portion 211, a curved portion 214, and a flexible portion 215. The distal end portion 211, the curved portion 214, and the flexible portion 215 can be connected in order from the distal end side. A channel 216 for inserting the treatment instrument 100 therethrough can be provided inside the insertion section 210. The distal end portion 211 can be provided with a distal end opening 212 of the channel 216 and an imaging unit 213.

The imaging unit 213 can include an imaging device such as a charged coupled device (CCD) or a complimentary metal-oxide semiconductor (CMOS), or any similar device capable of imaging a region to be treated. The curved portion 214 can bend according to or responsive to the user's operation of the operation section 220. The flexible portion 215 can be a flexible tubular portion.

The operation section 220 can be connected to the flexible portion 215. The operation section 220 can include a grip 221, an input unit 222, and a forceps opening 223. The grip 221 can be a member that is supported by the user. The input unit 222 can receive an operation for bending the curved portion 214. The forceps opening 223 can be a proximal end opening of the channel 216.

The universal cord 230 can connect the endoscope 200 and an external device. An imaging cable, an optical fiber cable, or the like, which outputs an imaging signal captured by the imaging unit 213 to the outside, can be inserted into the universal cord 230.

### Endoscopic Treatment Instrument 100

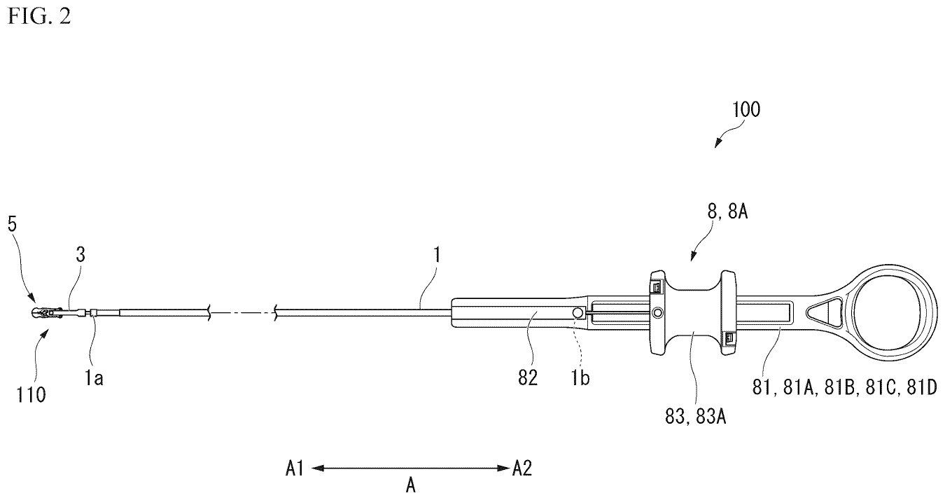

FIG. 2 is an example of an overall view illustrating the endoscopic treatment instrument 100.

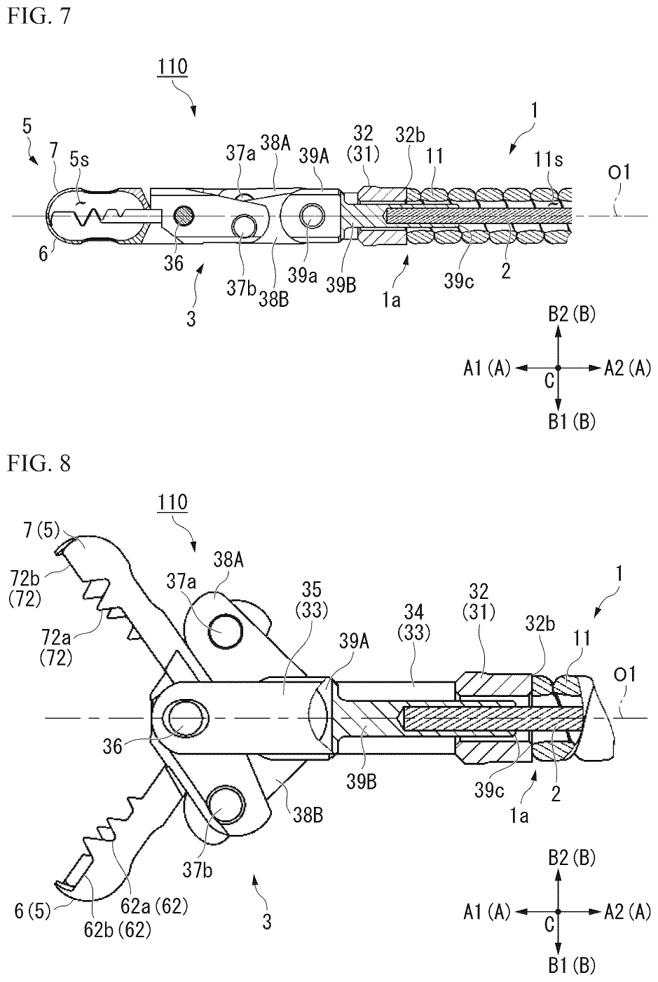

The endoscopic treatment instrument 100 (also referred to as the treatment instrument 100) can include a sheath 1, an operation wire 2 (see FIG. 7 below), a support member (e.g., a cover member) 3, forceps (e.g., jaws, arms) (end effector) 5, and a wire operating section 8. In the following description, as illustrated in FIG. 2, in the longitudinal direction “A” along the center axis of the treatment instrument 100, the side inserted into the patient's body is referred to as the “distal end side (distal side) A1” and the side of the wire operating section 8 is referred to as the “proximal end side (proximal side) A2”. The wire 2 can be provided in the coil sheath 1 and is configured to move in the longitudinal direction A of the coil sheath 1 relative to the coil sheath 1. The end effector 5 can be clip, snare, knife, needle, basket, balloon, stent, brash. In the case that the end effector does not open and close, the closed state corresponds to a state in which the operation wire 2 can be located at the most distal position, and the open state corresponds to a state in which operation wire 2 can be located at the most proximal position. The most distal position and the most proximal position can be located within a movable range of the operation wire 2.

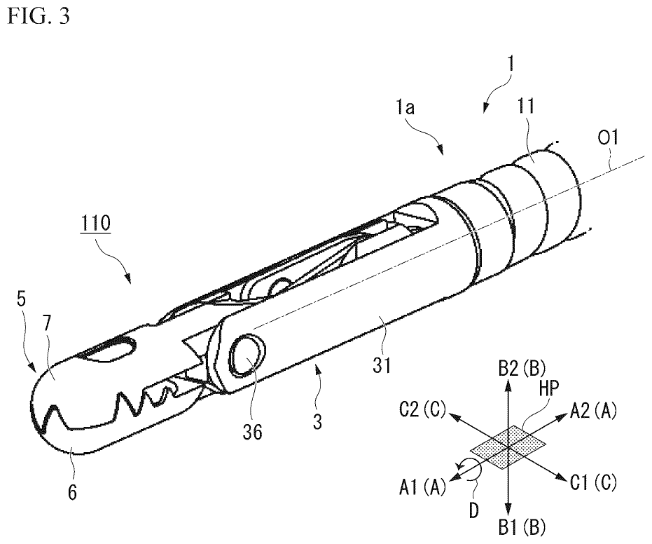

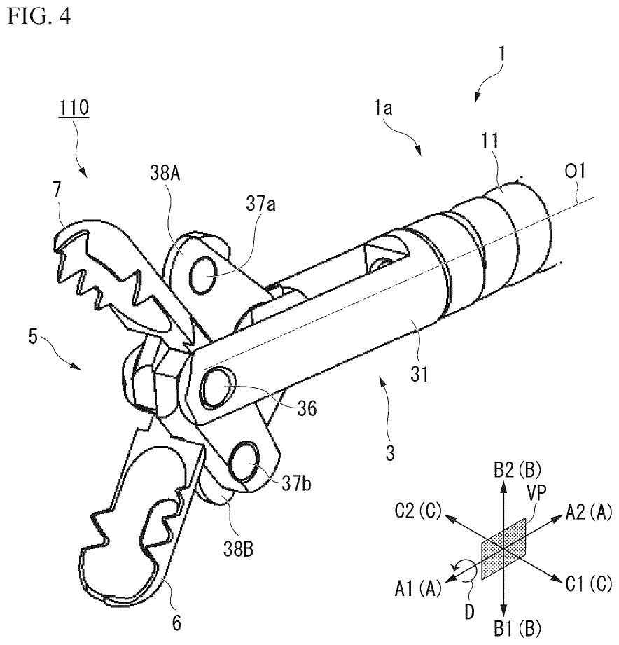

FIG. 3 is an example of a perspective view of the distal end portion of the treatment instrument 100 in a closed state. FIG. 4 is an example of a perspective view of the distal end portion of the treatment instrument 100 in an open state. As illustrated in FIG. 3, the distal end portion of the treatment instrument 100 can be provided with a support member 3 and the forceps 5. The forceps 5 can be supported by the support member 3 which can be a link mechanism to be openable and closable. The support member 3 and the forceps 5 constitute a “treatment section 110” that can treat an affected area. The end effector 5 can be configured to switch between the open state and the closed state in response to movement of the wire 2. When the end effector 5 is in the closed state, a proximal end of the wire connection portion 39 can be located proximally relative to a distal end of the coil sheath 1. When the end effector 5 is in the open state, the proximal end of the wire connection portion 39 can be located distally relative to the distal end of the coil sheath 1. A coil sheath connection portion 32 can be connected to a distal end side of the coil sheath 1. When the end effector 5 is in the closed state, the proximal end of the wire connection portion 39 can be located proximally relative to a proximal end of the coil sheath connection portion 32. When the end effector 5 is in the open state, the proximal end of the wire connection portion 39 can be located distally relative to the proximal end of the coil sheath connection portion 32. The end effector 5 can comprise a first arm 6 and a second arm 7. In the closed state, the first arm 6 of a plurality of arms is in contact with the second arm 7 or a distance between a distal end of the first arm and a distal end of the second arm can be zero. Zero can include a design error range of 1 mm. The end effector can be forceps.

In the following description, as illustrated in FIGS. 3 and 4, the direction in which the forceps 5 open and close is referred to as the “opening/closing direction B” or the “vertical direction B”. The direction in which a forceps piece 6 opens is referred to as the “lower side B1” in the opening/closing direction B and the direction in which a forceps piece 7 opens is referred to as the “upper side B2” in the opening/closing direction B.

Further, the direction perpendicular to the longitudinal direction A and the opening/closing direction B is referred to as the “width direction C” or the “horizontal direction C.” When a proximal end side A2 is viewed from a distal end side A1, the rightward direction is referred to as the “right side C1” in the width direction C and the leftward direction is referred to as the “left side C2” in the width direction C.

Further, as illustrated in FIG. 3, a plane which is horizontal in the longitudinal direction A and the width direction C is referred to as the “horizontal plane HP”. As illustrated in FIG. 4, a plane which is horizontal in the longitudinal direction A and the opening/closing direction B is referred to as the “vertical plane VP”.

The sheath 1 can be flexible and can be, as illustrated in FIGS. 1 and 2, an elongate member extending from a distal end la to a proximal end 1b. The sheath 1 can have an outer diameter allowing the sheath 1 to be inserted into the channel 216 of the endoscope 200. As illustrated in FIG. 1, the distal end la of the sheath 1 can protrude and retract from the distal end opening 212 of the channel 216 while the sheath 1 is inserted into the channel 216.

The sheath 1 can be a coil sheath formed by spirally winding a metal wire. Here, as illustrated in FIGS. 3 and 4, the sheath 1 can be formed by winding a wire 11 in the circumferential direction D of the longitudinal direction A. The coil sheath can comprise the winding wire, and the winding wire can have an elliptical cross-sectional shape. The winding wire can have a partially flat surface.

### Treatment Section 110

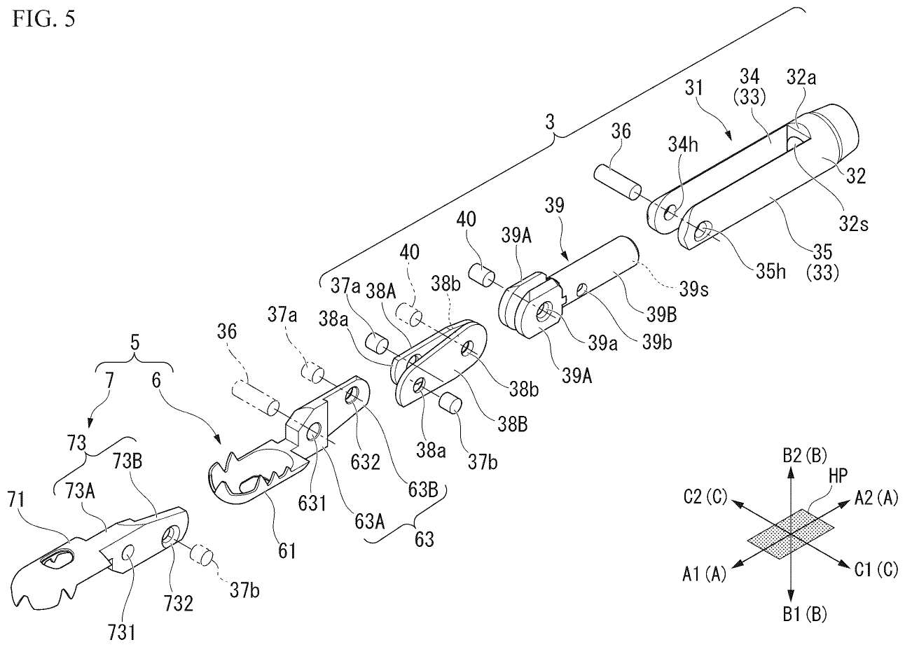

FIG. 5 is an example of an exploded perspective view of the treatment section 110.

As illustrated in FIG. 1, the support member 3 can support the forceps 5 at the distal end side A1 while the proximal end side A2 is attached to the distal end la of the sheath 1.

The support member 3 can include a frame 31, a forceps opening/closing pin (a rotation shaft and a rotation shaft member) 36, forceps connection pins 37a and 37b, a pair of support plate materials 38A and 38B, a connection portion 39, and a support plate connection pin 40. As illustrated in FIGS. 3 and 4, the forceps 5 can be connected to the forceps opening/closing pin 36 to be openable and closable around the axis of the forceps opening/closing pin 36 attached to the frame 31.

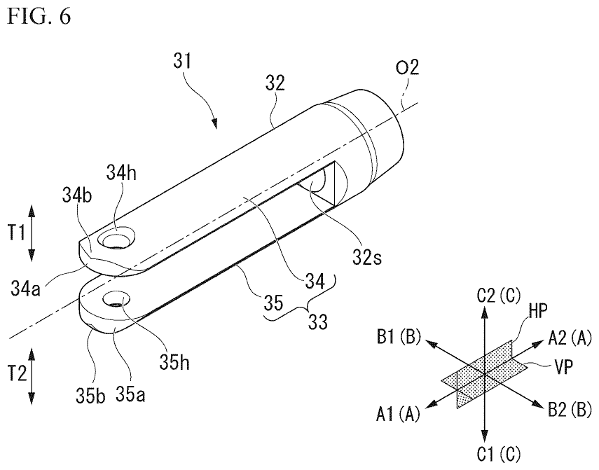

FIG. 6 is an example of a perspective view of the frame 31.

The frame 31 can be made of metal such as stainless steel and can have or be formed in a substantially U-shape. The frame 31 can include a support main body (coil sheath connection portion) 32 formed in a cylindrical shape and a pair of frame pieces 33 (a first frame piece 34 and a second frame piece 35). A center axis O2 of the support member 3 (frame 31) in the longitudinal direction, A, substantially coincides with a center axis O1 of the sheath 1 in the longitudinal direction A.

Furthermore, in this embodiment, the support main body 32 can be fixed to the distal end la of the sheath 1 by laser welding. Therefore, the rigid length can be shortened compared to bonding by conventional brazing.

As illustrated in FIG. 7, the pair of frame pieces 33 (the first frame piece 34 and the second frame piece 35) can be provided to protrude from the support main body 32 toward the distal end side A1. The first frame piece 34 and the second frame piece 35 can be equally provided on both sides of the horizontal direction C with the center axis O2 interposed therebetween. The first frame piece 34 and the second frame piece 35 can have or be formed in a symmetrical shape with respect to the vertical plane VP passing through the center axis O2.

The first frame piece 34 can be formed in a flat plate shape extending in the longitudinal direction A. The plate thickness direction T1 of the first frame piece 34 can substantially coincide with the horizontal direction C. The proximal end side A2 of the first frame piece 34 can be connected to the support main body 32.

A semicircular first distal end portion 34a can be formed on the distal end side A1 of the first frame piece 34. An inclined side 34b can be partially formed on the outside of the first distal end portion 34a in the width direction C. The thickness of the first frame piece 34 in the plate thickness direction T1 can be constant in the longitudinal direction A. A first through-hole 34h that penetrates in the plate thickness direction T1 can be formed on the distal end side A1 of the first frame piece 34.

The second frame piece 35 can be formed in a flat plate shape extending in the longitudinal direction A. The plate thickness direction T2 of the second frame piece 35 can substantially coincide with the horizontal direction C. The proximal end side A2 of the second frame piece 35 can be connected to the support main body 32.

A semicircular second distal end portion 35a can be formed on the distal end side A1 of the second frame piece 35. An inclined side 35b can be partially formed on the outside of the second distal end portion 35a in the width direction C. The thickness of the second frame piece 35 in the plate thickness direction T2 can be the same as the thickness of the first frame piece 34 in the plate thickness direction T1. A second through-hole 35h that penetrates in the plate thickness direction T2 can be formed on the distal end side A1 of the second frame piece 35. The center of the second through-hole 35h can coincide with the center of the first through-hole 34h.

The forceps opening/closing pin (the rotation shaft and the rotation shaft member) 36 can be made of metal such as stainless steel and has a substantially columnar shape. As illustrated in FIG. 5, the forceps opening/closing pin 36 can engage with the first through-hole 34h of the first frame piece 34 and the second through-hole 35h of the second frame piece 35 and can be attached to the frame 31.

The pair of support plate materials 38A and 38B can be formed in a plate shape by metal such as stainless steel. The pair of support plate materials 38A and 38B can have the same shape and can be overlapped in an upside-down orientation in the plate thickness direction. In each of the support plate materials 38A and 38B, a first through-hole 38a can be formed on the distal end side A1 and a second through-hole 38b can be formed on the proximal end side A2. The pair of support plate materials 38A and 38B can be overlapped in a state in which the second through-holes 38b on the proximal end side A2 can communicate or engage with each other and the first through-holes 38a can be offset from each other in the vertical direction B (the opening/closing direction of the forceps 5).

The support plate material 38A can be connected to the forceps piece (first forceps piece) 6 of the forceps 5 via the forceps connection pin 37a inserted through the first through-hole 38a. The support plate material 38B can be connected to the forceps piece (second forceps piece) 7 of the forceps 5 via the forceps connection pin 37b inserted through the first through-hole 38a. The support plate materials 38A and 38B can be connected to the connection portion 39 via the support plate connection pin 40 inserted through their second through-holes 38b and can be configured or supported to be rotatable around the axis of the support plate connection pin 40.

The connection portion (wire connection portion) 39 can include a pair of connection pieces 39A located on the distal end side A1 and a cylindrical body 39B, having a columnar shape (or cylindrical shape), and connected to the proximal end side A2 of the connection piece 39A. An inner space 39s can be formed in the cylindrical body 39B, the distal end side A1 is closed, and the proximal end side A2 is opened. A through-hole 39b, which communicates or engages with the inner space 39s from the right side C1 can be formed on the distal end side A1 of the cylindrical body 39B. The through-hole 39b can be formed so as to not penetrate to the left side C2 of the cylindrical body 39B. Furthermore, the through-hole 39b can be located so that it is not formed in the cylindrical body 39B. The wire connection portion 39 can be connected to a distal end side of the wire 2. The wire connection portion 39 can have a first outer diameter, the coil sheath can have a first inner diameter, and the first outer diameter can be smaller than the first inner diameter. The coil sheath connection portion 32 can be connected to a distal end side of the coil sheath 1, the coil sheath connection portion 32 can include a first portion and a second portion, the first portion can be located distally relative to the second portion. The first portion can have a first length between the center axis O1 of the coil sheath 1 and an outer surface of the coil sheath connection portion 32. The second portion can have a second length between the center axis O1 of the coil sheath 1 and the outer surface of the coil sheath connection portion 32, the second length can be smaller than the first length. The coil sheath 1 can include a third potion having a third length between the center axis O1 of the coil sheath 1 and an outer surface of the coil sheath 1. The third length can be equal to or smaller than the second length. The coil sheath 1 can include a fourth portion provided proximally relative to the third portion, the fourth portion can have a fourth length between the center axis O1 of the coil sheath 1 and the outer surface of the coil sheath 1. The fourth length can be smaller than the third length.

The pair of connection pieces 39A can be provided in parallel with a gap therebetween in the width direction C. The pair of support plate materials 38A and 38B can be inserted between the pair of connection pieces 39A. A through-hole 39a that penetrates in the plate thickness direction can be formed at the center of each connection piece 39A. The through-holes 39a can be concentric circles and communicate, engage, or connect with each other in the width direction C. The connection portion 39 can rotatably support the support plate materials 38A and 38B via the support plate connection pin 40 inserted through each of the through-holes 39a formed in the pair of connection pieces 39A. Furthermore, in this embodiment, the pair of connection pieces 39A, the support plate connection pin 40, and the support plate materials 38A and 38B can be configured or located so that they are not subjected to (or do not require) any bonding process such as caulking.

The cylindrical body 39B of the connection portion 39 can be inserted from the distal end side A1 of the frame 31 into an inner space 32s of the support main body 32 and the pair of connection pieces 39A are engaged to a distal end surface 32a of the support main body 32. The operation wire 2 to be described later can be connected to the cylindrical body 39B of the connection portion 39. Further, the sheath 1 can be connected to the support main body 32.

The forceps (jaws) 5 are members for collecting biological tissue. The forceps 5 can be made of a metal material such as stainless steel and include the first forceps piece 6 and the second forceps piece 7 which face each other the vertical direction B in a closed state. When the first forceps piece 6 and the second forceps piece 7 are in a closed state, the forceps 5 can have a cylindrical shape as illustrated in FIG. 3.

The first forceps piece 6 and the second forceps piece 7 can be supported by the pin 36 to be rotatable around the rotation axis of the pin 36. Furthermore, in this embodiment, the pin 36, the first frame piece 34, and the second frame piece 35 can be connected by laser welding. Therefore, the slidability between the pin 36 and the first and second forceps pieces 6 and 7 can be improved. The pin 36 can be connected to any one of the first frame piece 34 and the second frame piece 35 by laser welding. When the pin 36 is connected to any one of the first frame piece 34 and the second frame piece 35 by laser welding, the slidability between the pin 36 and the first and second forceps pieces 6 and 7 can be improved.

The first forceps piece 6 and the second forceps piece 7 can be respectively connected to the support plate materials 38A and 38B at the proximal end side A2. Further, the support plate materials 38A and 38B can be connected to the connection portion 39 and can be operated by the operation wire 2 connected to the connection portion 39. Furthermore, one of the first forceps piece 6 or the second forceps piece 7 can be fixed to the support member 3 and only the other thereof can be rotatably supported by the support member 3.

The first forceps piece 6 can be formed, for example, from a single flat plate or a single bar by mainly cutting or pressing. As illustrated in FIG. 5, the first forceps piece 6 can include a forceps cup (first forceps cup) 61 provided on the distal end side A1 of the longitudinal direction A and a first plate 63 can be provided on the proximal end side A2 of the longitudinal direction A.

FIG. 7 is an example of a cross-sectional view illustrating the distal end portion (treatment section 110) of the treatment instrument 100 in a closed state. FIG. 8 is an example of a cross-sectional view illustrating the distal end portion (treatment section 110) of the treatment instrument 100 in an open state.

The first forceps cup 61 can be formed in a substantially hemispherical shape and opens toward the second forceps piece 7 (upper side B2) in the opening/closing direction B. As illustrated in FIGS. 7 and 8, an opening edge 62 of the first forceps cup 61 can be provided with a plurality of protruding teeth 62a and recesses 62b corresponding to an unevenness shape in the opening/closing direction B in the closed forceps 5.

As illustrated in FIG. 5, the first plate 63 can provided on the proximal end side A2 of the first forceps cup 61 and has a substantially plate shape. The plate thickness direction of the first plate 63 can substantially coincide with the width direction C. The first plate 63 can be inserted into the frame 31 and can be disposed adjacent to the support plate material 38A on the left side C2 of the support plate material 38A. The first plate 63 can be rotatably connected to the support plate material 38A by the pin 37a. Furthermore, in this embodiment, the first plate 63, the pin 37a, and the support plate material 38A can be connected by caulking. Therefore, sufficient bonding strength can be ensured.

The first plate 63 can include a first portion 63A and a second portion 63B. The first portion 63A can be formed on the distal end side A1 of the first plate 63. The first portion 63A can be disposed adjacent to the second forceps piece 7 on the right side C1 of the first frame piece 34 of the frame 31 illustrated in FIG. 5 and can be located between the first frame piece 34 and a second plate 73 of the second forceps piece 7.

The second portion 63B can be formed on the proximal end side A2 of the first portion 63A. The second portion 63B can be disposed adjacent to the support plate material 38A on the right side C1 of the first frame piece 34 and can be located between the first frame piece 34 and the support plate material 38A. The thickness of the first portion 63A in the width direction C can be thicker than the thickness of the second portion 63B and a difference in thickness between the first portion 63A and the second portion 63B can be substantially the same as the plate thickness of the support plate material 38A.

The first plate 63 can include a first hole 631 and a second hole 632. The first hole 631 can be a through-hole that can penetrate in the plate thickness direction of the first portion 63A and can engage with, connect to, communicate with, or the like, the first through-hole 34h of the first frame piece 34 illustrated in FIG. 5. The second hole 632 can be a through-hole that can penetrate in the plate thickness direction of the second portion 63B and can engage with, connect to, communicates with, or the like, the first through-hole 38a of the support plate material 38A illustrated in FIG. 5.

As illustrated in FIG. 5, the pin 36 can be insertable through the first hole 631. Further, the pin 37a can be insertable through the second hole 632. The first portion 63A of the first forceps piece 6 can be rotatably connected to the frame 31 via the pin 36. The second portion 63B of the first forceps piece 6 can be rotatably connected to the support plate material 38A via the pin 37a.

The second forceps piece 7 can be formed, for example, from a single flat plate or a single bar by mainly cutting or pressing. As illustrated in FIG. 5, the second forceps piece 7 can include a forceps cup (second forceps cup) 71 provided on the distal end side A1 of the longitudinal direction A and a second plate 73 provided on the proximal end side A2 of the longitudinal direction A.

The second forceps cup 71 can be formed in a substantially hemispherical shape and can open toward the first forceps piece 6 (lower side B1) in the opening/closing direction B. As illustrated in FIGS. 7 and 8, an opening edge 72 of the second forceps cup 71 can be provided with a plurality of protruding teeth 72a and protrusions 72b corresponding to an unevenness shape in the opening/closing direction B in the closed forceps 5.

As illustrated in FIG. 5, the second plate 73 can be provided on the proximal end side A2 of the second forceps cup 71 and can have or be formed in a substantially plate shape. The plate thickness direction of the second plate 73 can substantially coincide with the width direction C. The second plate 73 can be inserted into the frame 31 and can be disposed adjacent to the support plate material 38B on the right side C1 of the support plate material 38B. The second plate 73 can be rotatably connected to the support plate material 38B by the pin 37b.

The second plate 73 can include a first portion 73A and a second portion 73B. The first portion 73A can be formed on the distal end side A1 of the second plate 73. The first portion 73A can be disposed adjacent to the first forceps piece 6 on the left side C2 of the second frame piece 35 of the frame 31 illustrated in FIG. 5 and can be located between the second frame piece 35 and the first plate 63 of the first forceps piece 6.

The second portion 73B can be formed on the proximal end side A2 of the first portion 73A. The second portion 73B can be disposed adjacent to the support plate material 38B on the left side C2 of the second frame piece 35 and can be located between the second frame piece 35 and the support plate material 38B. The thickness of the first portion 73A in the width direction C can be thicker than the thickness of the second portion 73B and a difference in thickness between the first portion 73A and the second portion 73B can be substantially the same as the plate thickness of the support plate material 38B.

The second plate 73 can include a first hole 731 and a second hole 732. The first hole 731 can be a through-hole that penetrates in the plate thickness direction of the first portion 73A and can engage with, connect to, communicate with, or the like, the second through-hole 35h of the second frame piece 35 illustrated in FIG. 5. The second hole 732 can be a through-hole that can penetrate in the plate thickness direction of the second portion 73B and can engage with, connect to, communicate with, or the like, the first through-hole 38a of the support plate material 38B illustrated in FIG. 5.

As illustrated in FIG. 5, the pin 36 can be insertable through the first hole 731. Further, the pin 37b can be insertable through the second hole 732. The first portion 73A of the second forceps piece 7 can be rotatably connected to the frame 31 via the pin 36. The second portion 73B of the second forceps piece 7 can be rotatably connected to the support plate material 38B via the pin 37b.

As illustrated in FIG. 7, the protruding teeth 62a and the recesses 62b of the first forceps piece 6 and the protruding teeth 72a and the protrusions 72b of the second forceps piece 7 can have a meshing shape in the closed forceps 5. Further, in the closed forceps 5, a forceps space 5s can be formed inside the first forceps piece 6 and the second forceps piece 7 meshing with each other.

The operation wire (wire) 2 can be a metallic wire and can be inserted through an inner space 11s of the sheath 1. The sheath 1 can be a coil sheath formed by winding the metallic wire 11 in a spiral shape as illustrated in FIG. 7. The proximal end of the operation wire 2 can be connected to the wire operating section 8. Further, in the cross section illustrated in FIG. 7, the coil sheath 1 can be formed by winding the wire 11. The winding wire 11 can be continuous in the longitudinal direction A. The first center axis can be unparallel with respect to the second center axis.

Further, the inner space 32s of the support main body 32 illustrated in FIGS. 5 and 6 can communicate with, engage with, connect to, or the like, the inner space 11s of the sheath 1. The operation wire 2 can be inserted through the inner space 32s of the support main body 32. The distal end of the operation wire 2 can be connected to the connection portion (wire connection portion) 39. Furthermore, in this embodiment, the distal end of the operation wire 2 and the connection portion 39 can be fixed by laser welding. Therefore, the rigid length can be shortened compared to bonding by conventional brazing.

The forceps 5 can have a configuration in which the first forceps piece 6 and the second forceps piece 7 rotate around the axis of the forceps opening/closing pin 36. The connection portion 39 can be connected to the first forceps piece 6 and the second forceps piece 7 on the proximal end side A2 via the support plate materials 38A and 38B, and when the operation wire 2 connected to the connection portion 39 is operated, the first forceps piece 6 and the second forceps piece 7 can rotate relative to each other in the opening/closing direction B to open and close the forceps 5.

The user can open and close the forceps 5 by advancing and retracting the operation wire 2 in the longitudinal direction A. The user can open the forceps 5 to be an open state by advancing the operation wire 2 in the longitudinal direction A in the closed forceps 5 illustrated in FIG. 7. On the other hand, in the opened forceps 5 illustrated in FIG. 8, the user can close the forceps 5 to be a closed state by retracting the operation wire 2 in the longitudinal direction A.

The user can grip the biological tissue using the forceps 5 by closing the first forceps piece 6 and the second forceps piece 7 while operating the operation wire 2 so that the opened forceps 5 can move toward the biological tissue. At this time, since the protruding teeth 62a and the recesses 62b of the first forceps piece 6 mesh with the protruding teeth 72a and the protrusions 72b of the second forceps piece 7, the biological tissue can be gripped without slipping. Further, since a part of the gripped biological tissue is accommodated in the forceps space 5s of the forceps 5, the operation of closing the forceps 5 can be suppressed from being obstructed by the gripped biological tissue, and the biological tissue can be stably gripped.

### Wire Operating Section 8

The wire operating section 8 can be provided, as illustrated in FIGS. 1 and 2, on the proximal end side A2 of the sheath 1. The wire operating section 8 can include an operating section main body 81, a main body lid 82, and a slider 83.

The operating section main body 81 can be connected to the proximal end 1b of the sheath 1. Specifically, the proximal end 1b of the sheath 1 can be provided in a groove, slot, or the like, formed in the operating section main body 81 along the longitudinal direction A and can be connected to the proximal end of the groove.

The main body lid 82 can be provided in the groove of the operating section main body 81 in an attachable and detachable manner. When the main body lid 82 is mounted on the operating section main body 81, the proximal end 1b of the sheath 1 can be fixed to the operating section main body 81.

The slider 83 can be attached to the operating section main body 81 to be movable in the longitudinal direction A. The proximal end of the operation wire 2 can be connected to the slider 83. When the user advances and retracts the slider 83 relative to the operating section main body 81, the operation wire 2 can advance and retract.

As illustrated in FIG. 7, in the closed treatment section 110, a proximal end 39c of the connection portion (wire connection portion) 39 can be located on the proximal end side A2 in relation to a proximal end 32b of the support main body (coil sheath connection portion) 32. At this time, it is possible to suppress the length of the support main body (coil sheath connection portion) 32 in the longitudinal direction A from becoming longer than necessary and to suppress the rigid length of the treatment section 110 from becoming longer.

Further, in the opened treatment section 110 illustrated in FIG. 8, the proximal end 39c of the connection portion (wire connection portion) 39 can be located on the distal end side A1 in relation to the proximal end 32b of the support main body (coil sheath connection portion) 32 and can be located in the inner space of the support main body (coil sheath connection portion) 32.

For example, when the length of the support main body 32 in the longitudinal direction A is longer than necessary and the rigid length of the treatment section 110 becomes long, in the curved insertion section 210 of the endoscope 200, the curvature radius of the treatment instrument 100 becomes large and the treatment instrument 100 can easily make contact with the channel 216 into which the treatment instrument 100 is inserted.

If the channel 216 and the treatment instrument 100 contact each other when the user advances and retracts the operation wire 2 to grip the biological tissue by operating the wire operating section 8 and removes the treatment instrument 100 gripping the biological tissue from the endoscope 200, a large force can be required to remove the treatment instrument 100 and operability can be reduced.

In the closed treatment section 110, when the proximal end 39c of the connection portion (wire connection portion) 39 is located on the proximal end side A2 in relation to the proximal end 32b of the support main body (coil sheath connection portion) 32, it is possible to suppress an increase in the rigid length of the treatment instrument 100, suppress the treatment instrument 100 from contacting the channel 216 in the curved insertion section 210, and improve the operability of the treatment instrument 100.

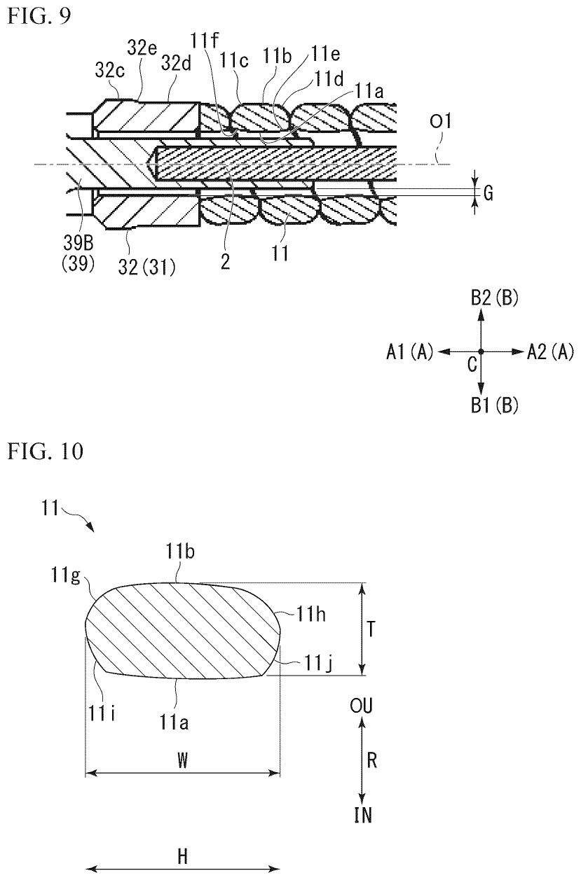

FIG. 9 is an example of a cross-sectional view illustrating a connection portion between the operation wire 2 and the cylindrical body 39B in the closed treatment section 110. FIG. 10 is an example of a cross-sectional view illustrating the wire 11 that forms the sheath (coil sheath) 1.

Here, the direction orthogonal to the longitudinal direction A of the sheath 1 is defined as the “radial direction R”. Further, in the radial direction R, the direction moving close to the center axis O1 of the sheath 1 is referred to as the “inside IN” and the direction moving away from the center axis O1 is defined as the “outside OU”.

A surface (inner surface, inclined inner surface) 11a on the inside IN of the wire 11 forming the coil sheath 1 can form the inner peripheral surface of the coil sheath 1. A proximal end 11e (second inner surface) of the inner surface 11a of the wire 11 can be closer to the center axis O1 of the coil sheath 1 than a distal end 11f (first inner surface) of the inner surface 11a. That is, the inner surface 11a of the wire 11 can be inclined so that the proximal end 11e is closer to the center axis O1 than the distal end 11f. Here, the proximal end 11e and the distal end 11f of the inner surface 11a of the wire 11 are not the proximal end and the distal end of the entire coil sheath 1 and can be the proximal end 11e and the distal end 11f of the wire 11 in the width direction in the cross section of the wire 11 as illustrated in FIG. 9. The coil sheath 1 can include the first inner surface 11f and the second inner surface 11e located proximally relative to the first inner surface. The second inner surface can be closer to the center axis O1 of the coil sheath 1 than the first inner surface. The coil sheath can include an inclined inner surface 11a inclined relative to a center axis of the coil sheath. The inclined inner surface 11a can face distally. The inclined inner surface 11a can comprise the first inclined inner surface 11f. The coil sheath 1 can include the second inclined inner surface 11e facing distally. The first inclined inner surface 11a can oppose to the second inclined inner surface 11a with respect to the center axis O1 of the coil sheath 1. The coil sheath 1 can include a plurality of inclined inner surfaces 11a.

When closing the opened forceps 5, the operation wire 2 and the connection portion (wire connection portion) 39 connected to the distal end of the operation wire 2 can retract through the inner space 11s of the coil sheath 1 toward the proximal end side A2. At this time, for example, when the proximal end 39c of the connection portion 39 contacts the corner portion on the distal end side A1 of the inner surface 11a of the wire 11, the connection portion 39 can get caught by the wire 11 and can prevent the operation wire 2 from retracting. As a result, the operability of the treatment instrument 100 can be reduced.

In the cross section illustrated in FIG. 9, since the coil sheath 1 can be formed by connecting the wire 11, inclined so that the proximal end 11e of the inner surface 11a is closer to the center axis O1 than the distal end 11f, in the longitudinal direction A as described above, the proximal end 39c of the connection portion 39 retracting toward the proximal end side A2 does not contact the corner portion on the distal end side A1 of the inner surface 11a of the wire 11. For example, when the proximal end 39c of the connection portion 39 contacts the inner surface 11a of the wire 11, the proximal end 39c of the connection portion 39 can contact the proximal end 11e of the inner surface 11a of the wire 11. Therefore, the connection portion 39 can retract without being caught by the wire 11 and the operability of the treatment instrument 100 can be improved. The coil sheath comprises a first inclined inner surface 11a and a second inclined inner surface 11a. The first inclined inner surface 11a can face distally. The first inclined inner surface 11f can include a first inner surface 11a and a second inner surface 11e can be located proximally relative to the first inner surface 11f. The second inner surface 11e is located closer to the center axis O1 than the first inner surface 11a. The second inclined inner surface 11a can face distally. The second inclined inner surface 11a can include a third inner surface 11f and a fourth inner surface 11e located proximally relative to the third inner surface. The fourth inner surface 11e can be located closer to the center axis O1 than the third inner surface 11f. The second inner surface 11e is closer to the center axis O1 than the third inner surface 11f. The coil sheath 1 can include a first outer surface and a second outer surface located proximally relative to the first outer surface, and the second outer surface can be located closer to the center axis O1 than the first outer surface. The coil sheath 1 can include the inclined outer surface, the inclined outer surface can face proximally.

Further, in a surface (e.g., an outer surface) 11b on the outside OU of the wire 11 forming the outer peripheral surface of the coil sheath 1, a proximal end 11d of the outer surface 11b of the wire 11 can be closer to the center axis O1 of the coil sheath 1 than a distal end 11c of the outer surface 11b. That is, the outer surface 11b of the wire 11 can be inclined so that the proximal end 11d is closer to the center axis O1 than the distal end 11c. Here, the proximal end 11d and the distal end 11c of the outer surface 11b of the wire 11 are not the proximal end and the distal end of the entire coil sheath 1 and can be the proximal end 11d and the distal end 11c of the wire 11 in the width direction in the cross section of the wire 11 as illustrated in FIG. 9.

In this way, the coil sheath 1 can be formed by connecting the wire 11, inclined so that the proximal end is closer to the center axis O1 than the distal end in the inner surface 11a and the outer surface 11b, in the longitudinal direction A. For example, the coil sheath 1 can be formed by winding the wire 11 of which the distal end side A1 and the proximal end side A2 can have a symmetrical shape with respect to the center in the longitudinal direction A in such a manner that the wire can be inclined so that the proximal end is closer to the center axis O1 than the distal end.

Further, as illustrated in FIG. 9, the support main body 32 can include a large diameter portion 32c provided on the distal end side A1 and a small diameter portion 32d provided on the proximal end side A2 of the large diameter portion 32c. In the outer peripheral surface of the support main body 32 having a cylindrical shape extending around the center axis O1, the diameter of the small diameter portion 32d can be smaller than that of the large diameter portion 32c. As illustrated in FIG. 9, a step portion 32e can be provided between the large diameter portion 32c and the small diameter portion 32d.

Here, the distance from the center axis O1 to the distal end 11c of the outer surface 11b of the wire 11 can be the same as the distance from the center axis O1 to the proximal end of the small diameter portion 32d. That is, in the outer peripheral surfaces of the support main body (coil sheath connection portion) 32 and the coil sheath 1, the proximal end 11d of the outer surface 11b of the wire 11 can be closer to the center axis 01 than the distal end 11c of the outer surface 11b, the distal end 11c of the outer surface 11b and the small diameter portion 32d can have the same distance from the center axis

O1, and the small diameter portion 32d can be closer to the center axis O1 than the large diameter portion 32c.

Therefore, since the outer peripheral surfaces of the support main body 32 and the coil sheath 1 are less likely to be caught by the inner peripheral surface of the channel 216 when removing the treatment instrument 100 from the endoscope 200, the operability of the treatment instrument 100 can be improved.

Furthermore, the positional relationship between the distal end 11c of the outer surface 11b of the wire 11, the large diameter portion 32c, and the small diameter portion 32d is not limited thereto.

The distance from the center axis O1 to the distal end 11c of the outer surface 11b of the wire 11 can be the same as the distance from the center axis O1 to the large diameter portion 32c. Even in this case, since the outer peripheral surfaces of the support main body 32 and the coil sheath 1 are less likely to be caught by the inner peripheral surface of the channel 216 when removing the treatment instrument 100 from the endoscope 200, the operability of the treatment instrument 100 can be improved.

Further, the distal end 11c of the outer surface 11b of the wire 11 can be closer to the center axis O1 than the large diameter portion 32c and the small diameter portion 32d can be closer to the center axis O1 than the distal end 11c of the outer surface 11b of the wire 11. Even in this case, since the outer peripheral surfaces of the support main body 32 and the coil sheath 1 are less likely to be caught by the inner peripheral surface of the channel 216 when removing the treatment instrument 100 from the endoscope 200, the operability of the treatment instrument 100 can be improved.

Further, the distance G between the support main body (coil sheath connection portion) 32 and the cylindrical body 39B of the connection portion (wire connection portion) 39 in the radial direction R illustrated in FIG. 9 can be 0.02 mm or more and 0.2 mm or less. Furthermore, at this time, the center axis of the support main body 32 and the center axis of the connection portion 39 can coincide with the center axis O1 of the coil sheath 1. Further, when the coil sheath 1 is not curved, the distance between the support main body 32 and the inner peripheral surface of the coil sheath 1 in the radial direction R can coincide with the distance G. The distance G can be 0.04 mm or more and 0.1 mm or less. The distance G between the coil sheath 1 and the wire connection portion 39 in a radial direction can be 0.02 mm or more and 0.2 mm or less. The wire 2 can have a first diameter, and the wire connection portion can have a second diameter larger than the first diameter.

When the insertion section 210 is curved, the coil sheath 1 inserted into the channel 216 can also be curved. Since the connection portion (wire connection portion) 39 formed of a rigid member is not curved as much as the coil sheath 1 when the coil sheath 1 is curved, there is a possibility that the connection portion 39 can inhibit the bending operation of the coil sheath 1.

When the distance G is set to 0.02 mm or more and 0.2 mm or less and the gap between the outer peripheral surface of the connection portion 39 and the inner peripheral surface of the support main body 32 can be sufficiently set, the operation of bending the coil sheath 1 can be suppressed from being obstructed by the connection portion 39 compared to the case in which the gap is not sufficient and hence the operability of the treatment instrument 100 can be improved. That is, the substantial rigid length of the connection portion 39 can be made shorter compared to the case in which the gap is not sufficient.

Next, various dimensions in the cross-sectional shape of the wire 11 will be described with reference to FIG. 10.

As illustrated in FIG. 10, the outer shape of the wire 11 can be formed by the inner surface 11a, the outer surface 11b, a first corner portion 11g, a second corner portion 11h, a third corner portion 11i, and a fourth corner portion 11j.

As described above, the inner surface 11a can be a surface located on the inside IN closer to the center axis O1 in the coil sheath 1 formed by winding the wire 11 in a spiral shape.

As described above, the outer surface 11b can be a surface located on the outside OU of the radial direction R in the coil sheath 1 formed by winding the wire 11 in a spiral shape.

As illustrated in FIG. 10, the first corner portion 11g and the second corner portion 11h can be surfaces connected to the outer surface 11b. The distal end 11c of the outer surface 11b illustrated in FIG. 9 can be a boundary portion in which the outer surface 11b and the first corner portion 11g are connected. Further, the proximal end 11d of the outer surface 11b illustrated in FIG. 9 can be a boundary portion in which the outer surface 11b and the second corner portion 11h are connected.

As illustrated in FIG. 10, the third corner portion 11i and the fourth corner portion 11j can be surfaces connected to the inner surface 11a. The third corner portion 11i can be connected to the first corner portion 11g. The fourth corner portion 11j can be connected to the second corner portion 11h.

The first corner portion 11g, the second corner portion 11h, the third corner portion 11i, and the fourth corner portion 11j can be rounded and have a rounded shape. The shape of the wire 11 can be formed by rolling or drawing.

The R dimension (radius) of the inner surface 11a can be 0.30 mm or more. The inner surface 11a can have a flat surface.

The R dimension of the outer surface 11b can be R equal to 0.3 mm or more. The outer surface 11b can have a flat surface or a concave surface.

The R dimensions of the first corner portion 11g and the second corner portion 11h can be R equal to 0.05 mm or more and 0.30 mm or less. The first corner portion 11g, the second corner portion 11h, and the outer surface 11b are smoothly connected without any edge at the boundary portion where each surface is connected.

The R dimensions of the third corner portion 11i and the fourth corner portion 11j can be R equal to 0.05 mm or more and 0.30 mm or less.

Further, the thickness T of the wire 11 can be 0.15 mm or more and 0.40 mm or less. The width W of the wire 11 can be 0.20 mm or more and 0.80 mm or less and can be 1.33 times or more and 2.0 times or less the thickness T. Here, as illustrated in FIG. 10, the thickness T, and the width W of the wire 11 are dimensions in the orthogonal directions.

Furthermore, as described above, the wire 11 forming the coil sheath 1 can be wound while being inclined so that the proximal end is closer to the center axis O1 than the distal end in the inner surface 11a and the outer surface 11b. Therefore, the radial direction R of the coil sheath 1 and the direction indicating the thickness T of the wire 11 do not necessarily match. In the cross section illustrated in FIG. 10, for convenience of description, the radial direction R matches the direction indicating the thickness T.

The endoscopic treatment instrument 100 according to this embodiment can include the coil sheath (sheath) 1 formed by winding the wire 11 in a spiral shape, the coil sheath connection portion (support main body) 32 which can be connected to the distal end of the coil sheath 1, the wire (operation wire) 2 which can be inserted through the coil sheath 1 and can advance and retract in the longitudinal direction A of the coil sheath 1, the forceps 5 which include the first forceps piece 6 and the second forceps piece 7 and can be supported to be openable and closable toward the distal end side A1 in the longitudinal direction A, and the wire connection portion (connection portion) 39 which is connected to the distal end of the wire 2 and is inserted through the coil sheath connection portion 32 to open and close the forceps 5.

Further, when the forceps 5 are in a closed state, the proximal end 39c of the wire connection portion 39 can be located on the proximal end side A2 of the longitudinal direction A in relation to the proximal end 32b of the coil sheath connection portion 32. Therefore, it can be possible to suppress an increase in the rigid length of the endoscopic treatment instrument 100 and to suppress the contact between the channel 216 and the endoscopic treatment instrument 100 in the curved insertion section 210.

As a result, the endoscopic treatment instrument 100 having improved operability can be provided.

Further, in a surface (inner surface 11a) on the inside IN of the wire 11 forming the inner peripheral surface of the coil sheath 1, the proximal end 11e of the inner surface 11a can be closer to the center axis O1 of the coil sheath 1 than the distal end 11f of the inner surface 11a. Therefore, since it can be possible to suppress the connection portion 39 connected to the distal end of the operation wire 2 from being caught by the distal end 11f of the inner surface 11a of the wire 11 when retracting the operation wire 2, it is possible to improve the operability of the endoscopic treatment instrument 100.

Further, in a surface (outer surface 11b) on the outside OU of the wire 11 forming the outer peripheral surface of the coil sheath 1, the proximal end 11d of the outer surface 11b can be closer to the center axis O1 of the coil sheath 1 than the distal end 11c of the outer surface 11b.

Furthermore, the coil sheath connection portion 32 can include the large diameter portion 32c which can be provided on the distal end side A1 and the small diameter portion 32d which can be provided on the proximal end side A2 of the large diameter portion 32c and can have a smaller diameter than the large diameter portion 32c. The distance from the center axis O1 to the distal end 11c of the outer surface 11b can be the same as the distance from the center axis O1 to the proximal end of the small diameter portion 32d.

Therefore, since the outer peripheral surfaces of the coil sheath connection portion 32 and the coil sheath 1 are less likely to be caught by the inner peripheral surface of the channel 216 when removing the endoscopic treatment instrument 100 from the endoscope 200, the operability of the endoscopic treatment instrument 100 can be improved.

Further, the distance G (gap) between the coil sheath connection portion 32 and the wire connection portion 39 in the radial direction R can be 0.02 mm or more and 0.2 mm or less. Therefore, since the gap between the outer peripheral surface of the wire connection portion 39 and the inner peripheral surface of the coil sheath connection portion 32 can be sufficiently set, the operation of bending the coil sheath 1 can be suppressed from being obstructed by the wire connection portion 39 compared to the case in which the gap is not sufficient and hence the operability of the endoscopic treatment instrument 100 can be improved.

In this way, in the endoscopic treatment instrument 100 according to this embodiment, the coil sheath 1 and the channel 216 can slide smoothly without getting caught and sliding resistance can be reduced. Accordingly, it is possible to reduce the force (inserting force) when inserting the endoscopic treatment instrument 100 into the channel 216, the force (tearing force) when tearing off the biological tissue with the forceps 5, and the force (removing force) when removing the endoscopic treatment instrument 100 from the channel 216 and to improve the operability of the endoscopic treatment instrument 100.

Although the first embodiment of the present invention has been described above in detail with reference to the drawings, the specific configuration is not limited to this embodiment, and design changes may be made within the scope of the present disclosure. Further, the components illustrated in the above-described embodiments and modified examples can be configured by appropriately combining them.

### First Modified Example

The shape and configuration of the wire operating section 8 are not limited to the embodiments illustrated in FIGS. 1 and 2.

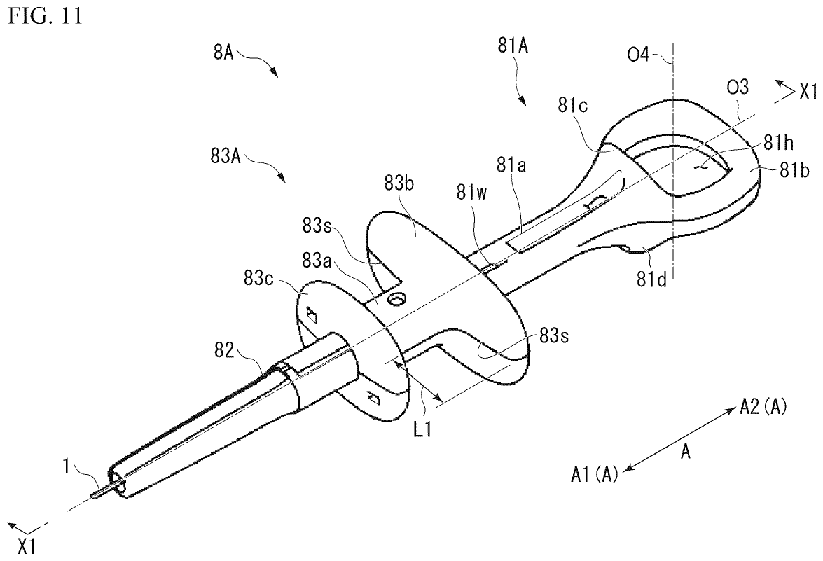

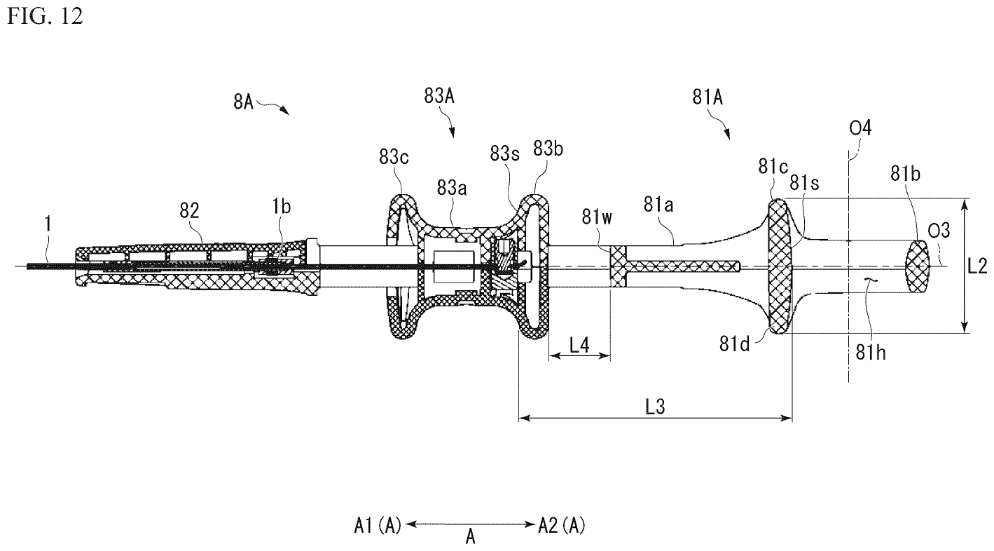

FIG. 11 is an example of a perspective view illustrating a wire operating section 8A according to a first modified example. FIG. 12 is an example of a cross-sectional view illustrating the wire operating section 8A according to the first modified example. In the following description, components that are common to those already described will be given the same reference numerals and redundant description will be omitted.

The wire operating section 8A can include an operating section main body 81A, a main body lid 82, and a slider 83A. As illustrated in FIG. 12, the proximal end 1b of the sheath 1 can be fixed to the operating section main body 81A. Further, the main body lid 82 can be provided in the operating section main body 81A in an attachable and detachable manner.

As in the wire operating section 8 of the above-described embodiment, the wire operating section 8A can be an operation section that advances and retracts the operation wire 2 connected to the slider 83A by advancing and retracting the slider 83A, attached to the operating section main body 81A to be movable in the longitudinal direction A, relative to the operating section main body 81A.

The operating section main body 81A can include a main body portion 81a, a first gripping portion 81b, a first grip extension portion 81c, and a second grip extension portion 81d.

The main body portion 81a can be a substantially columnar member that extends along a center axis O3 of the wire operating section 8A. The center axis O3 of the wire operating section 8A can coincide with the longitudinal direction A.

The first gripping portion 81b can be provided at the proximal end of the main body portion 81a and can have or be formed in a substantially U shape opening toward the distal end side A1. Both ends of the first gripping portion 81b on the proximal end side A2 are connected to the proximal end of the main body portion 81a. A gripping space 81h can penetrate in a direction along an axis O4 and can be formed in a space surrounded by the proximal end of the main body portion 81a and the first gripping portion 81b. The gripping space 81h can be a space in which the user who operates the wire operating section 8A mainly places his or her thumb.

Further, the first grip extension portion 81c and the second grip extension portion 81d can extend in a direction along the axis O4 and can be provided at the proximal end of the main body portion 81a. The first grip extension portion 81c and the second grip extension portion 81d can protrude in a direction moving away from the center axis O3 from the proximal end of the main body portion 81a in a direction along the axis O4. The first grip extension portion 81c and the second grip extension portion 81d can protrude in different directions.

A surface on the proximal end side A2 of the first grip extension portion 81c and a surface on the proximal end side A2 of the second grip extension portion 81d can be smoothly connected to form a first gripping surface 81s. The first gripping surface 81s can be a surface that the user who operates the wire operating section 8A mainly contacts with the pad of the thumb.

The slider 83A can include a slider main body 83a, a second gripping portion 83b, and a third gripping portion 83c. The slider main body 83a is a portion that can be operated mainly by the index finger and middle finger of the user who operates the wire operating section 8A.

The slider main body 83a can have a substantially cylindrical shape that extends around the center axis O3. The second gripping portion 83b can be connected to the proximal end of the slider main body 83a and can protrude in a direction moving away from the center axis O3 and the outer shape of the slider main body 83a. Specifically, as illustrated in FIG. 11, the second gripping portion 83b can protrudes in a direction orthogonal to the center axis O3 and the axis O4 and a second gripping surface 83s can be formed on the distal end side A1 of the second gripping portion 83b.

The second gripping surface 83s can be a surface that the user who operates the wire operating section 8A mainly contacts with the pads of the index finger and middle finger when moving the slider 83A toward the proximal end side A2 with respect to the operating section main body 81A. At this time, the user can place his or her index and middle fingers so as to sandwich the slider main body 83a between them.

The third gripping portion 83c can be connected to the distal end of the slider main body 83a and can protrude in a direction moving away from the center axis O3 and the outer shape of the slider main body 83a. The third gripping portion 83c can be a surface that the user who operates the wire operating section 8A mainly contacts with his or her index finger and middle finger when moving the slider 83A toward the distal end side A1 with respect to the operating section main body 81A. At this time, the user can bring the nails or the vicinity of the nails on the side opposite to the pads of the index and middle fingers into contact with the surface on the proximal end side A2 of the third gripping portion 83c.

Next, each dimension of the wire operating section 8A will be described. The dimension L1 illustrated in FIG. 11 can be a dimension from the outer peripheral surface of the slider main body 83a to the outer periphery of the second gripping portion 83b in the second gripping surface 83s. This is not limited to the case in which the connection portion between the slider main body 83a and the second gripping surface 83s or the outer periphery of the second gripping surface 83s is chamfered or rounded, and the dimension L1 can be a dimension (width) of a portion with which the user who operates the wire operating section 8A can contact the pads of the index finger and middle finger.

The dimension L1 can be 10 mm or more and 12 mm or less. In an example, dimension L1 can be 11.7 mm.

The dimension L2 illustrated in FIG. 12 can be a dimension of the first gripping surface 81s in a direction along the axis O4. Specifically, the dimension can be obtained by adding the dimension of the first grip extension portion 81c in a direction along the axis O4 to the dimension of the second grip extension portion 81d in a direction along the axis O4. This is not limited to the case in which the outer edges of the first grip extension portion 81c and the second grip extension portion 81d are chamfered or rounded, and the dimension L2 can be a dimension (width) of a portion that the user who operates the wire operating section 8A can make contact with the pad of the thumb.

The dimension L2 can be 22 mm or more and 24 mm or less. In an example, the dimension L2 can be 23.9 mm.

The dimension L3 illustrated in FIG. 12 can be a dimension indicating the distance from the first gripping surface 81s to the second gripping surface 83s in the longitudinal direction A. For example, the dimension L3 can be a distance from the pad of the thumb to the pads of the index and middle fingers of the user's right hand when the user operates the wire operating section 8A with his or her right hand and moves the slider 83A toward the proximal end side A2 with respect to the operating section main body 81A.

The dimension L3 can be 40 mm or more. Since it is easy to apply a force to the wire operating section 8A when the user operates the wire operating section 8A by setting the dimension L3 to 40 mm or more, the operability can be improved. By setting the dimensions L1, L2, and L3 as described above, the grippable force (e.g., the gripping force) of the wire operating section 8A can be improved, and the amount of biological tissue collected by the forceps 5 can be improved.

The dimension L4 illustrated in FIG. 12 can be a movement amount (stroke amount) in which the slider 83A is movable toward the proximal end side A2 with respect to the operating section main body 81A when the opened forceps 5 are operated to be in a closed state. When the opened forceps 5 are moved to a closed state, the slider 83A can be moved toward the proximal end side A2 with respect to the operating section main body 81A. At this time, the proximal end of the slider 83A can collide with a regulation surface 81w illustrated in FIGS. 11 and 12.

The dimension L4 can affect the amount of the tensile force (the amount of the tensile force for the wire connection portion) when retracting the wire connection portion 39 connected to the distal end of the operation wire 2. Here, the dimension L4 can be set such that the amount of the tensile force at the wire connection portion is 40 N or less. Since parts that are subjected to load (for example, the wire connection portion 39 and the operation wire 2) are suppressed from being damaged or broken when the operation wire 2 is retracted by setting the amount of the tensile force at the wire connection portion to 40 N or less, it is possible to suppress parts from falling off due to breakage.

### Second Modified Example

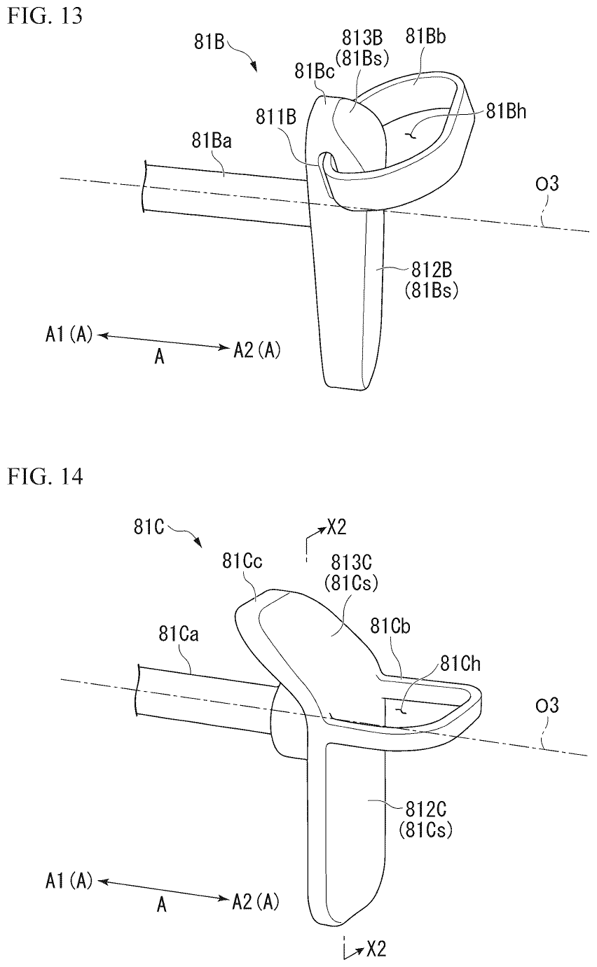

The shape and configuration of the operating section main bodies 81 and 81A are not limited to the embodiments illustrated in FIGS. 1, 2, 11, and 12. FIG. 13 is an example of a perspective view illustrating an operating section main body 81B according to a second modified example.

The operating section main body 81B can include a main body portion 81Ba, a first gripping portion 81Bb, and a first grip extension portion 81Bc.

The main body portion 81Ba can have a substantially columnar shape that extends around the center axis O3. The first grip extension portion 81Bc can be connected to the proximal end of the main body portion 81Ba. The first grip extension portion 81Bc can be or include a plate-shaped member that extends in a direction orthogonal to the extending direction of the center axis O3 and can include a through-hole 811B as illustrated in FIG. 13.

As illustrated in FIG. 13, the first gripping portion 81Bb having an annular shape can be inserted through the through-hole 811B. Further, a gripping space 81Bh can be formed in a space surrounded by the first gripping portion 81Bb and the first grip extension portion 81Bc.

A surface on the proximal end side A2 of the first grip extension portion 81Bc can be provided with a first gripping surface 81Bs. The first gripping surface 81Bs can include a flat portion 812B and an inclined portion 813B. The flat portion 812B and the inclined portion 813B are not on the same plane but can be arranged so that the first gripping surface 81Bs has a bent shape as illustrated in FIG. 13.

The first gripping surface 81Bs can be a surface with which the user contacts the pad of the thumb. For example, the user can bring a portion of the thumb closer to the distal end than the first joint into contact with the inclined portion 813B and can bring a portion of the thumb closer to the proximal end (palm side) than the first joint into contact with the flat portion 812B.

FIG. 14 is an example of a perspective view illustrating an operating section main body 81C according to the second modified example.

The operating section main body 81C can include a main body portion 81Ca, a first gripping portion 81Cb, and a first grip extension portion 81Cc.

The main body portion 81Ca can have a substantially columnar shape that extends around the center axis O3. The first grip extension portion 81Cc can be connected to the proximal end of the main body portion 81Ca. A surface on the proximal end side A2 of the first grip extension portion 81Cc can be provided with a first gripping surface 81Cs. The first gripping surface 81Cs includes a flat portion 812C and an inclined portion 813C. The flat portion 812C and the inclined portion 813C are not on the same plane but can be arranged so that the first gripping surface 81Cs has a bent shape as illustrated in FIG. 14.

The first gripping portion 81Cb can be a substantially U-shaped member that opens on the distal end side A1. Both ends of the first gripping portion 81Cb on the distal end side A1 can be connected to the first gripping surface 81Cs. The first gripping portion 81Cb can be connected to the flat portion 812C of the first gripping surface 81Cs.

A gripping space 81Ch can be formed in a space surrounded by the first gripping portion 81Cb and the first gripping surface 81Cs.

The first gripping surface 81Cs can be a surface with which the user contacts the pad of the thumb. For example, the user can bring a portion of the thumb closer to the distal end than the first joint into contact with the inclined portion 813C and can bring a portion of the thumb closer to the proximal end (palm side) than the first joint into contact with the flat portion 812C.

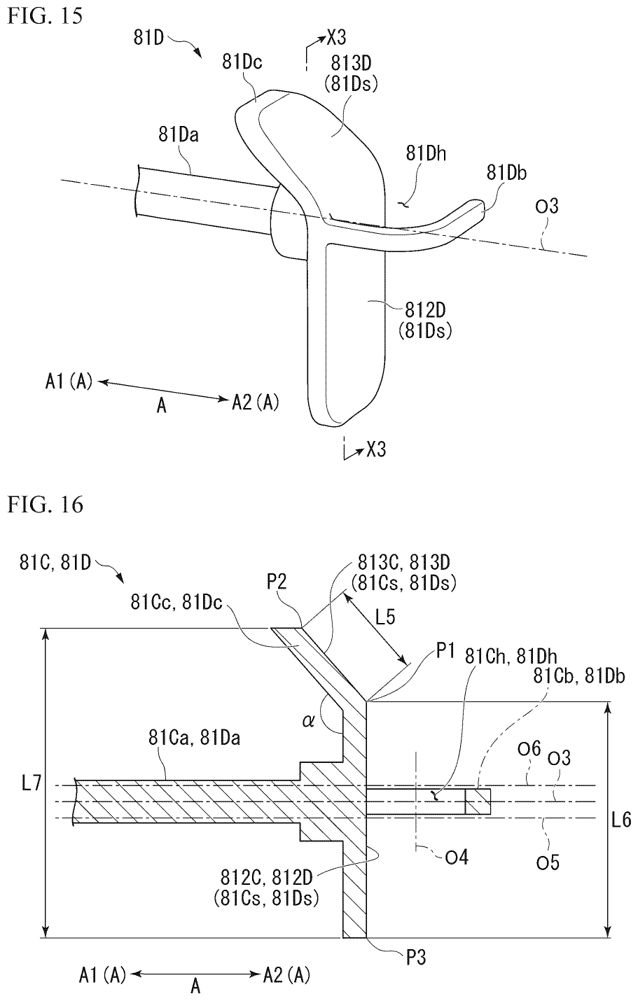

FIG. 15 is an example of a perspective view illustrating an operating section main body 81D according to the second modified example.

The operating section main body 81D can include a main body portion 81Da, a first gripping portion 81Db, and a first grip extension portion 81Dc.

The main body portion 81Da can have a substantially columnar shape that can extend around the center axis O3. The first grip extension portion 81Dc can be connected to the proximal end of the main body portion 81Da. A surface on the proximal end side A2 of the first grip extension portion 81Dc can be provided with a first gripping surface 81Ds. The first gripping surface 81Ds can include a flat portion 812D and an inclined portion 813D. The flat portion 812D and the inclined portion 813D are not on the same plane but can be arranged so that the first gripping surface 81Ds has a bent shape as illustrated in FIG. 15.

The first gripping portion 81Db can be a substantially L-shaped member having a portion extending in parallel to the center axis O3 and a portion extending perpendicularly to the direction in which the center axis O3 extends and the direction in which the flat portion 812D extends. The distal end of the first gripping portion 81Db can be connected to the first gripping surface 81Ds. The first gripping portion 81Db can be connected to the flat portion 812D of the first gripping surface 81Ds.

A gripping space 81Dh can be formed in a space sandwiched between the first gripping portion 81Db and the first gripping surface 81Ds.

The first gripping surface 81Ds can be a surface with which the user contacts the pad of his or her thumb. For example, the user can bring a portion of the thumb closer to the distal end than the first joint into contact with the inclined portion 813D and a portion of the thumb closer to the proximal end (palm side) than the first joint into contact with the flat portion 812D.

FIG. 16 is an example of a cross-sectional view illustrating the operating section main bodies 81C and 81D according to the second modified example.

The dimension L5 illustrated in FIG. 16 can be a dimension indicating the distance from a boundary P1 corresponding to a boundary portion between the flat portions 812C and 812D and the inclined portions 813C and 813D and end portions P2 of the inclined portions 813C and 813D. That is, the dimension L5 can be a dimension indicating the length of the inclined portions 813C and 813D.

The dimension L6 illustrated in FIG. 16 can be a dimension indicating the distance between the boundary P1 and the end portions P3 of the flat portions 812C and 812D. That is, the dimension L6 can be a dimension indicating the length of the flat portions 812C and 812D. Furthermore, the dimension L6 can be a dimension in a direction perpendicular to the center axis O3 in the cross section illustrated in FIG. 16.

Further, the dimension L6 can be a dimension in a direction along the axes O4 of the gripping spaces 81Ch and 81Dh.

Here, for example, the dimension L6 indicating the length of the flat portions 812C and 812D can be 2.5 times or more and 5.0 times or less the dimension L5 indicating the length of the inclined portions 813C and 813D.

The dimension L7 illustrated in FIG. 16 can be a dimension indicating the distance between the end portions P2 of the inclined portions 813C and 813D and the end portions P3 of the flat portions 812C and 812D. Furthermore, the dimension L7 can be a dimension in a direction perpendicular to the center axis O3 in the cross section illustrated in FIG. 16. Further, the dimension L7 can be a dimension in a direction along the axes O4 of the gripping spaces 81Ch and 81Dh.

The center axis O5 illustrated in FIG. 16 can be the center axis of the dimension L6. The center axis O5 can extend in parallel to the center axis O3.

The center axis O6 illustrated in FIG. 16 can be the center axis of the dimension L7. The center axis O6 can extend in parallel to the center axis O3 and the center axis O6.

Here, the center axes O3 of the main body portions 81Ca and 81Da can be located between the center axis O5 of the dimension L6 and the center axis O6 of the dimension L7.

Further, the angle a illustrated in FIG. 16 indicates an angle of a corner formed by the flat portions 812C and 812D and the inclined portions 813C and 813D. Here, the angle a can be 130° or more and 160° or less.

### Third Modified Example

Not only when the forceps 5 are in a closed state, the proximal end 39c of the wire connection portion 39 can be located on the proximal end side A2 of the longitudinal direction A in relation to the proximal end 32b of the coil sheath connection portion 32.

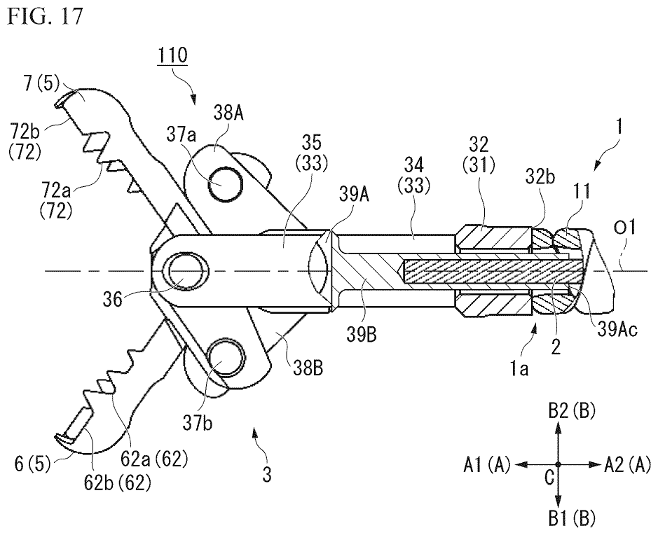

FIG. 17 is an example of a cross-sectional view illustrating the distal end portion (treatment section 110) of the endoscopic treatment instrument 100 in an open state according to a modified example embodiment. In FIG. 17, when the forceps 5 are in a closed state, a proximal end 39Ac of the wire connection portion 39 can be located on the proximal end side A2 of the longitudinal direction A in relation to the proximal end 32b of the coil sheath connection portion 32. When the end effector 5 is in the open state, the proximal end of the wire connection portion 39 is located proximally relative to the proximal end of the coil sheath connection portion 32.

Even if when the forceps 5 are in a closed state, and a proximal end 39Ac of the wire connection portion 39 can be located on the proximal end side A2 of the longitudinal direction A in relation to the proximal end 32b of the coil sheath connection portion 32, it can be possible to suppress an increase in the rigid length of the endoscopic treatment instrument 100 and the operability of the treatment instrument 100 can be improved.

Example 1. An endoscopic treatment instrument comprising:
a coil sheath which is formed by winding a wire in a spiral shape;
a coil sheath connection portion which is connected to a distal end of the coil sheath;
a wire which is inserted through the coil sheath and can advance and retract in a longitudinal direction of the coil sheath;
forceps which include a first forceps piece and a second forceps piece and are supported to be openable and closable toward the distal end side in the longitudinal direction; and
a wire connection portion which is connected to a distal end of the wire and is inserted through the coil sheath connection portion to open and close the forceps,
wherein when the forceps are in a closed state, a proximal end of the wire connection portion is located on a proximal end side of the longitudinal direction in relation to a proximal end of the coil sheath connection portion.

Example 2. The endoscopic treatment instrument according to Example 1,
wherein when the forceps are in an open state, the proximal end of the wire connection portion is located on the distal end side in relation to the proximal end of the coil sheath connection portion.

Example 3. The endoscopic treatment instrument according to Example 1,
wherein in a surface on the inside of the wire forming an inner peripheral surface of the coil sheath, a proximal end of the wire in a width direction is closer to a center axis of the coil sheath than a distal end of the wire in the width direction.

Example 4. The endoscopic treatment instrument according to Example 3,
wherein the coil sheath connection portion has a substantially cylindrical shape extending around the center axis and includes a large diameter portion which is provided on the distal end side and a small diameter portion which is provided on the proximal end side and has a smaller diameter than the large diameter portion, and
wherein in a surface on the outside of the wire forming an outer peripheral surface of the coil sheath, the proximal end of the wire in the width direction is closer to the center axis than the distal end of the wire in the width direction.

Example 5. The endoscopic treatment instrument according to Example 4,
wherein in a surface on the outside of the wire forming the outer peripheral surface of the coil sheath, the distance from the center axis to the distal end of the wire in the width direction is substantially the same as the distance from the center axis to a proximal end of the small diameter portion.

Example 6. The endoscopic treatment instrument according to Example 3,
wherein the wire connection portion has a substantially columnar shape extending around the center axis, and
wherein when a center axis of the coil sheath connection portion is substantially the same as a center axis of the wire connection portion, the distance between the coil sheath connection portion and the wire connection portion in a radial direction is 0.02 mm or more and 0.2 mm or less.

Example 7. The endoscopic treatment instrument according to Example 6,
wherein the distance between the coil sheath connection portion and the wire connection portion in the radial direction is 0.04 mm or more and 0.1 mm or less.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. An endoscopic treatment instrument comprising:
a coil sheath;
a wire provided in the coil sheath and configured to move in a longitudinal direction of the coil sheath relative to the coil sheath;
a wire connection portion connected to a distal end side of the wire; and
an end effector configured to switch between an open state and closed state in response to movement of the wire, wherein when the end effector is in the closed state, a proximal end of the wire connection portion is located proximally relative to a distal end of the coil sheath.

2. The endoscopic treatment instrument according to claim 1, wherein when the end effector is in the open state, the proximal end of the wire connection portion is located distally relative to the distal end of the coil sheath.

3. The endoscopic treatment instrument according to claim 1, further comprising:
a coil sheath connection portion connected to a distal end side of the coil sheath, wherein when the end effector is in the closed state, the proximal end of the wire connection portion is located proximally relative to a proximal end of the coil sheath connection portion.

4. The endoscopic treatment instrument according to claim 3, wherein when the end effector is in the open state, the proximal end of the wire connection portion is located distally relative to the proximal end of the coil sheath connection portion.

5. The endoscopic treatment instrument according to claim 1, wherein when the end effector is in the open state, the proximal end of the wire connection portion is located proximally relative to the proximal end of the coil sheath connection portion.

6. The endoscopic treatment instrument according to claim 1, wherein the coil sheath includes a first inner surface and a second inner surface located proximally relative to the first inner surface, and wherein the second inner surface is closer to a center axis of the coil sheath than the first inner surface.

7. The endoscopic treatment instrument according to claim 1, wherein the coil sheath includes an inclined inner surface inclined relative to a center axis of the coil sheath, and wherein the inclined inner surface faces distally.

8. The endoscopic treatment instrument according to claim 7, wherein the inclined inner surface comprises a first inclined inner surface, wherein the coil sheath includes a second inclined inner surface facing distally, and wherein the first inclined inner surface opposes to the second inclined inner surface with respect to a center axis of the coil sheath.

9. The endoscopic treatment instrument according to claim 6, wherein the coil sheath includes a plurality of inclined inner surfaces.

10. The endoscopic treatment instrument according to claim 1, wherein the coil sheath comprises:
a first inclined inner surface facing distally, wherein the first inclined inner surface includes a first inner surface and a second inner surface located proximally relative to the first inner surface, wherein the second inner surface is located closer to a center axis of the coil sheath than the first inner surface; and
a second inclined inner surface facing distally, wherein the second inclined inner surface includes a third inner surface and a fourth inner surface located proximally relative to the third inner surface, wherein the fourth inner surface is located closer to the center axis than the third inner surface, and wherein the second inner surface is closer to the center axis than the third inner surface.

11. The endoscopic treatment instrument according to claim 1, wherein the wire connection portion has a first outer diameter, wherein the coil sheath has a first inner diameter, and wherein the first outer diameter is smaller than the first inner diameter.

12. The endoscopic treatment instrument according to claim 1, wherein a distance between the coil sheath and the wire connection portion in a radial direction is 0.02 mm or more and 0.2 mm or less.

13. The endoscopic treatment instrument according to claim 1, wherein the wire has a first diameter, and wherein the wire connection portion has a second diameter larger than the first diameter.

14. The endoscopic treatment instrument according to claim 1, wherein the end effector comprises a first arm and a second arm,
in the closed state, the first arm is in contact with the second arm or a distance between a distal end of the first arm and a distal end of the second arm is zero.

15. The endoscopic treatment instrument according to claim 1, wherein the end effector is forceps.

16. The endoscopic treatment instrument according to claim 1, wherein the coil sheath comprises a winding wire, and wherein the winding wire has an elliptical cross-sectional shape.

17. The endoscopic treatment instrument according to claim 1, wherein the coil sheath includes a first outer surface and a second outer surface located proximally relative to the first outer surface, and wherein the second outer surface is located closer to a center axis of the coil sheath than the first outer surface.

18. The endoscopic treatment instrument according to claim 1, wherein the coil sheath includes an inclined outer surface, wherein the inclined outer surface faces proximally to a proximal end of the coil sheath.

19. The endoscopic treatment instrument according to claim 1, further comprising:
a coil sheath connection portion connected to a distal end side of the coil sheath,
wherein the coil sheath connection portion includes a first portion and a second portion, wherein the first portion is located distally relative to the second portion,
wherein the first portion has a first length between a center axis of the coil sheath and an outer surface of the coil sheath connection portion, and
wherein the second portion has a second length between the center axis of the coil sheath and the outer surface of the coil sheath connection portion, the second length is smaller than the first length, wherein, and
wherein the coil sheath includes a third potion having a third length between the center axis of the coil sheath and an outer surface of the coil sheath, wherein the third length is equal to or smaller than the second length.

20. The endoscopic treatment instrument according to claim 19, wherein the coil sheath includes a fourth portion provided proximally relative to the third portion, the fourth portion has a fourth length between the center axis of the coil sheath and the outer surface of the coil sheath, wherein the fourth length is smaller than the third length.
